(19) United States
(12) Patent Application Publication
(10) Pub. No.: US 2024/0280493 A1
(43) Pub. Date: Aug. 22, 2024

(54) CELLULAR SENSORS BASED ON SURFACE-ENHANCED RAMAN SCATTERING AND APPLICATION THEREOF

(71) Applicant: CHINA PHARMACEUTICAL UNIVERSITY, Nanjing, Jiangsu (CN)

(72) Inventors: Wenyuan LIU, Nanjing (CN); Lingfei HAN, Nanjing (CN); Feng FENG, Nanjing (CN)

(73) Assignee: CHINA PHARMACEUTICAL UNIVERSITY, Nanjing, Jiangsu (CN)

(21) Appl. No.: 18/570,741

(22) PCT Filed: Feb. 9, 2023
(86) PCT No.: PCT/CN2023/075166

§ 371 Date: Dec. 15, 2023

(30) Foreign Application Priority Data
May 26, 2022 (CN) 202210577889.4

(51) Int. Cl. G01N 21/65; B22F 1/054; B22F 1/102; B22F 1/145; B22F 9/24; G01N 33/50; G01N 33/543

(52) U.S. Cl. CPC G01N 21/658; B22F 1/054; B22F 1/102; B22F 1/145; B22F 9/24; G01N 33/5067; G01N 33/54346; B22F 2301/255; B22F 2304/054; B22F 2998/10; B22F 2999/00

(57) ABSTRACT

A cell-based sensor based on surface-enhanced Raman scattering and an application thereof. The cell-based sensor is established by guiding a surface-enhanced Raman scattering probe into a human liver cell line; the surface-enhanced Raman scattering probe is prepared by using a gold nanoparticle as a testing substrate, a gene damage effector molecule antibody as a recognition unit, a Raman molecule as a reporting unit, SH-PEG-NH₂ as a stable chain and a cell-penetrating peptide as an auxiliary penetration unit; and the cell-based sensor is exposed to various drug impurities, a Raman signal is detected, and a type and a level of the genotoxic impurities are assessed.

### TECHNICAL FIELD

The present disclosure belongs to the field of pharmaceutical analysis and testing and relates to a cell-based sensor based on surface-enhanced Raman scattering (SERS) and an application thereof in genotoxic impurity assessment.

### BACKGROUND

Performing toxicity assessment and limit control on various impurities possibly generated in various links of pharmaceutical research and development and clinical use is a significant requirement for ensuring drug quality and safety. Genotoxic impurities (GTI) are a type of impurities capable of causing gene damage and having a carcinogenic risk under a low concentration, and a drug regulatory administration in each nation has made a strict limit standard¹⁻² for a content of GTI in drugs. With development of a modern analytical technology, impurities (such as N-nitrosodimethylamine and methyl methanesulfonate) with known genotoxicity can be effectively tested and controlled. However, how to rapidly and effectively assess genotoxicity of impurities with unknown genotoxicity has become a key bottleneck issue.

Existing conventional GTI assessment methods³⁻⁵ (such as a rodent carcinogenicity test, an Ames test and a quantitative structure-activity relationship (QSAR)) and methods such as intra-cell molecular in-situ hybridization and DNA adduct testing applied in basic research are still confronted with many limits, which are shown as follows:
(1) animal test impurities are large in quantity, high in cost and long in period;
(2) a test based on a prokaryotic cell is largely different from a human cell and is not suitable for GTI assessment needing metabolic activation;
(3) being false positive/false negative is prone to occurring based on virtual screening methods such as a computer;
(4) rapid assessment of various gene damages are difficult to implement through assessment methods such as molecular hybridization or adduct testing for a single gene mutation site or a damage type; and
(5) other methods needing separation or dyeing are complicated in operation, cannot implement in-situ real-time testing, and may miss or interfere with a response process of an organism to a gene damage.

Therefore, the present disclosure establishes a testing platform based on human liver cells in vitro, using a common effector molecule after gene damage as a target and implementing in-situ on-line testing of an intracellular effector molecule, which is an effective strategy of solving the above technology bottleneck issue.

### REFERENCE DOCUMENTATION

1 Szekely G, Amores De Sousa M C, Gil M, Castelo Ferreira F, Heggie W. Genotoxic Impurities in Pharmaceutical Manufacturing: Sources, Regulations, and Mitigation. Chemical Reviews 2015; 115:8182-8229.
2 ICH guideline M7 on assessment and control of DNA reactive (mutagenic) impurities in pharmaceuticals to limit potential carcinogenic risk
3 Dearfield K L, Thybaud V, Cimino M C et al. Follow-up actions from positive results of in vitro genetic toxicity testing. Environmental and Molecular Mutagenesis 2011; 52:177-204.
4 Guo X, Seo J, Li X, Mei N. Genetic toxicity assessment using liver cell models: past, present, and future. Journal of Toxicology and Environmental Health, Part B 2020; 23:27-50.
5 Zhang Yuying, Li Wei, Pan Weisong. Overview on drug impurity genotoxicity assessment. Drug Evaluation 2021; 18:203-207.

### SUMMARY

The present disclosure provides a cell-based sensor based on surface-enhanced Raman scattering (SERS) and an application thereof in genotoxic impurity assessment in order to overcome defects of an existing genotoxicity assessment method. The present disclosure uses a gold nanoparticle as a testing substrate, a gene damage effector molecule antibody as a recognition unit and a Raman molecule as a reporting unit to prepare a SERS probe, and guides the probe into a human liver cell to establish a cell-based sensor. When a gene damage occurs, an effector molecule is overexpressed at the damage, probes are induced to aggregate to form a hotspot, a SERS enhanced signal is generated, in-situ real-time monitoring is performed under a Raman microscope, and impurity genotoxicity is assessed through intensity change of a Raman signal in a gene damage process, which is significant to pushing pharmaceutical research and development and ensuring drug safety.

Objectives of the present disclosure may be implemented through the following technical solutions:
a cell-based sensor based on surface-enhanced Raman scattering, which is established by guiding a surface-enhanced Raman scattering probe into a human liver cell line;
the surface-enhanced Raman scattering (SERS) probe is prepared by using a gold nanoparticle as a testing substrate, a gene damage effector molecule antibody as a recognition unit, a Raman molecule as a reporting unit, SH-PEG-NH₂ as a stable chain and a cell-penetrating peptide as an auxiliary penetration unit; and
the gene damage effector molecule antibody is a γH2AX antibody.

The Raman molecule includes at least one of 4-cyanothiophenol, 4-mercaptobenzoic acid or 4-mercaptophenylboronic acid.

The human liver cell line is at least one of a human liver cell L02, a human liver cancer cell HepG2 or a human liver cancer cell Hepa1-6.

The cell-penetrating peptide includes at least one of TAT or NLS.

As a preferred technical solution, the surface-enhanced Raman scattering probe also uses SH-PEG-NH₂ as a stable chain and a cell-penetrating peptide as an auxiliary penetration unit.

Further preferably, the surface-enhanced Raman scattering probe is prepared by the following steps:
step (1): preparing a gold nanoparticle (GNP) solution through a trisodium citrate reduction method;
step (2): modifying the gold nanoparticle by SH-PEG-NH₂, specifically, SH-PEG-NH₂ is added into the gold nanoparticle solution prepared in step (1) for a stirring reaction to obtain an SH-PEG-NH₂ modified gold nanoparticle solution;
step (3): modifying the gold nanoparticle by the Raman molecule, specifically, a Raman molecule solution is slowly added into the gold nanoparticle solution prepared in step (2) for a stirring reaction, then centrifugation is performed to remove a supernatant, and ultrapure water is added for uniform dispersion to obtain SH-PEG-NH₂ and a Raman molecule modified gold nanoparticle solution (GPM);
step (4): modifying the gold nanoparticle by the gene damage effector molecule antibody, specifically, 5% (5 g/100 ml) glutaraldehyde solution is added into the gold nanoparticle solution prepared in step (3) for a stirring reaction, then centrifugation is performed to remove a supernatant, ultrapure water is added for uniform dispersion to obtain a glutaraldehyde gold nanoparticle solution, then a gene damage effector molecule antibody aqueous solution is added for incubation, then centrifugation is performed to remove a supernatant, and ultrapure water is added for uniform dispersion to obtain a gene damage effector molecule antibody modified gold nanoparticle solution; and step (5): modifying the gold nanoparticle by the cell-penetrating peptide, specifically, the cell-penetrating peptide is added into the gold nanoparticle solution prepared in step (4) for a stirring reaction, then centrifugation is performed to remove a supernatant, PBS containing 1% BSA (1 g/100 ml) is used for redissolving, and uniform dispersion is performed to obtain a surface-enhanced Raman scattering probe (Anti γH2AX@GPMT).

Further preferably, a particle diameter of the gold nanoparticle is 10 to 50 nm.

Further preferably, a process of preparing the gold nanoparticle solution by using the trisodium citrate reduction method in step (1): a 0.01% (0.01 g/100 ml) HAuCl₄ aqueous solution is heated to boiling, 1% (1 g/100 ml) trisodium citrate aqueous solution is quickly added, and boiling is performed for 7 to 10 min, where a volume ratio of the 0.01% HAuCl₄ aqueous solution to the 1% trisodium citrate aqueous solution is 20:1 to 100:1.

Further preferably, a molecular weight of the SH-PEG-NH₂ in step (2) is 2000 to 5000; a molar ratio of the gold nanoparticle to the SH-PEG-NH₂ is 1:1×10³ to 1:2×10⁶;
the Raman molecule solution in step (3) is 1 mg/ml Raman molecule ethanol solution; a molar ratio of the gold nanoparticle to the Raman molecule is 1:1×10³ to 1:1×10⁶;
a molar ratio of the gold nanoparticle to the glutaraldehyde in step (4) is 1:1×10³ to 1:2×10⁶; a charge ratio of the gold nanoparticle to the gene damage effector molecule antibody is 5 pmol:2 μL to 5 nmol:2 μL; and
a molar ratio of the gold nanoparticle to the cell-penetrating peptide in step (5) is 1:1×10² to 1×1:10⁵.

Further preferably, time of each stirring reaction in step (2), step (3) and step (5) is 5 to 10 hours independently; and time of the stirring reaction in step (4) is 1 to 3 hours, and an incubation condition is incubation for 1 to 3 hours at 25° C. to 38° C.

A preferred technical solution of the preparation method of the above surface-enhanced Raman scattering (SERS) probe includes the following steps:
step (1): preparing the gold nanoparticle by using the trisodium citrate reduction method, specifically, a 0.01% (0.01 g/100 mL) HAuCl₄ aqueous solution is heated to boiling, a 1% (1 g/100 mL) trisodium citrate aqueous solution is quickly added, and boiling is performed for 7 to 10 min; where a volume ratio of the 0.01% HAuCl₄ aqueous solution to the 1% trisodium citrate aqueous solution is 20.1 to 100:1, and a particle diameter of the obtained gold nanoparticle is 10 to 50 nm;
step (2): modifying the gold nanoparticle by the SH-PEG-NH₂, specifically, the SH-PEG-NH₂ is added into the gold nanoparticle solution prepared in step (1), and stirring is performed for 5 to 10 h (most preferably, 6 h) to obtain an SH-PEG-NH₂ modified gold nanoparticle solution; where a molecular weight of the SH-PEG-NH₂ is 2000 to 5000, a molar ratio of the gold nanoparticle to the SH-PEG-NH₂ is 1:1×10³ to 1:2×10⁶, most preferably, 1:2×10⁴;
step (3): modifying the gold nanoparticle by the Raman molecule, specifically, a 1 mg/ml Raman molecule ethanol solution is slowly added into the gold nanoparticle solution prepared in step (2), stirring is performed for 5 to 10 h (most preferably, 6 h), 6000 rpm centrifugation is performed for 10 min to remove a supernatant, and uniform ultrasonic dispersion is performed with ultrapure water to obtain SH-PEG-NH₂ and a Raman molecule modified gold nanoparticle solution (GPM); where the Raman molecule includes at least one of 4-cyanothiophenol, 4-mercaptobenzoic acid or 4-mercaptophenylboronic acid; a molar ratio of the gold nanoparticle to the Raman molecule is 1:1×10² to 1:1×10⁶, preferably, 1:1×10⁵;
step (4): modifying the gold nanoparticle by the gene damage effector molecule antibody, specifically, a 5% (5 g/100 mL) glutaraldehyde solution is added into the gold nanoparticle solution (GPM) prepared in step (3), stirring is performed for 1 to 3 h (most preferably, 2 h), 6000 rpm centrifugation is performed for 10 min to remove a supernatant, ultrasonic dispersion is performed with ultrapure water to obtain a glutaraldehyde gold nanoparticle solution, then a gene damage effector molecule antibody aqueous solution is added, incubation is performed for 1 to 3 h (most preferably, 2 h) at 25° C. to 38° C., 6000 rpm centrifugation is performed for 10 min to remove a supernatant, and uniform ultrasonic dispersion is performed with ultrapure water to obtain a gene damage effector molecule antibody modified gold nanoparticle solution, where a molar ratio of the gold nanoparticle to the glutaraldehyde is 1:1×10³ to 1:2×10⁶, preferably, 1:2×10⁵; the gene damage effector molecule antibody is a γH2AX antibody; a charge ratio of the gold nanoparticle to the gene damage effector molecule antibody is 5 pmol:2 μL to 5 nmol:2 μL, preferably, 500 pmol:2 μL; and the gene damage effector molecule antibody is a regular commercial product; and
step (5): modifying the gold nanoparticle by the cell-penetrating peptide, specifically, the cell-penetrating peptide is added into the gold nanoparticle solution prepared in step (4), stirring is performed for 5 to 10 h (most preferably, 6 h), 6000 rpm centrifugation is performed for 10 min to remove a supernatant, PBS containing 1% BSA (1 g/100 mL) is used for redissolving, and uniform ultrasonic dispersion is performed to obtain a surface-enhanced Raman scattering probe (Anti γH2AX@GPMT); where the cell-penetrating peptide includes at least one of TAT or NLS; and a molar ratio of the gold nanoparticle to the cell-penetrating peptide is 1:1×10² to 1:1×10⁵, preferably, 1:1×10³.

An application of the above cell-based sensor in genotoxic impurity assessment.

A genotoxic impurity assessment method based on surface-enhanced Raman scattering uses a gold nanoparticle as a testing substrate, a gene damage effector molecule antibody as a recognition unit, a Raman molecule as a reporting unit, SH-PEG-NH₂ as a stable chain and a cell-penetrating peptide as an auxiliary penetration unit to prepare a surface-enhanced Raman scattering probe; the surface-enhanced Raman scattering probe is guided into a human liver cell line to establish a cell-based sensor; the cell-based sensor is exposed to drug impurities of different DNA damage mechanisms, a Raman signal is detected, and a genotoxicity level of the drug impurities is assessed; and the cell-based sensor is the above cell-based sensor.

Through the above method, when a gene damage occurs, an effector molecule is overexpressed at the damage, surface-enhanced Raman scattering probes are induced to aggregate to form a hotspot, a surface-enhanced Raman scattering enhanced signal is generated, in-situ real-time monitoring is performed under a Raman microscope, and a genotoxicity level of drug impurities is assessed through intensity change of a Raman signal in a gene damage process.

A specific operation process of the method includes the following steps: (1) establishing the cell-based sensor: the human liver cell is inoculated into a 24-well plate containing a cell slide, after being attached to a wall, a culture medium is replaced by an SERS probe culture medium with a concentration being 0.01 to 1 nM, and incubation is performed for 1 to 4 h. (2) Genotoxic impurity assessment: a cell-based sensor culture medium is replaced by a culture medium containing drug impurities of different concentrations, continuous incubation is performed for 24 h, then the slide is taken out and put in a confocal inverted microscope bright and dark field imaging Raman spectrometer to be tested, Raman signals of an test group and a control group are compared, and the genotoxicity level of the drug impurities is assessed.

Through the above method, a γH2AX cell-based sensor assesses a chromosome elastogen; the concentration of the drug impurities is supposed to ensure that a cell viability of the cell-based sensor is 75% or above; the confocal inverted microscope bright and dark field imaging Raman spectrometer needs to be equipped with a dark field condenser, a Raman spectrometer and other elements; when the Raman signal is detected, an excitation wavelength of a light source of the Raman spectrometer is 638 nm, and the detected signal uses a peak value of a characteristic Raman peak after 1800 cm⁻¹ of a Raman shift; and the detected signal is transformed to an effector molecule concentration through a standard curve, a concentration ratio of effector molecules of the test group and the control group is calculated, namely, a fold of induction (FI), when FI is greater than 1.5, it is judged as a DNA-damage type genotoxic impurity, and when FI is smaller than or equal to 1.5, it is judged as a non-DNA-damage type genotoxic impurity.

Through the technical solution of the present disclosure, a liver cell line containing rich metabolic enzymes is used as a carrier, and the human liver cell has a large quantity of metabolic enzyme systems, can simulate a vivo environment to the greatest degree and avoids some GTI omissions needing metabolic activation; the common effector molecule can respond to various types of gene damages at the same time and effectively improve a genotoxicity assessment speed; and organism gene damage and repair usually occur within a finite time, and in-situ real-time testing provides instant response information after a living cell gene damage, omits a complicated post-processing process and can effectively reduce a false negative rate or a false positive rate.

Anchoring the common effector molecule after the gene damage is a basis for improving universality of the method in the present disclosure. The genotoxic impurity damage mainly includes two types of a DNA damage and a cell division damage, most of existing genotoxic impurity damage types are DNA damages, including DNA alkylation, DNA crosslinking, single-stranded breakage, double-stranded breakage and the like, and each specific mechanism has a particular damage marker. γH2AX is a phosphorylation product of a histone H2AX, double-stranded breakage finally caused by DNA damages of different mechanisms causes γH2AX to be high-expressed around damaged DNA within a short time, and the γH2AX has become a universal biomarker of the DNA damages; and the present disclosure uses the γH2AX as a gene damage effector molecule to establish a GTI assessment system.

Intracellular in-situ real-time testing for effector molecule local high expression is another key issue to be solved by the present disclosure. The surface-enhanced Raman scattering (SERS) is a stable and nondestructive molecular spectrum testing technology, colloidal metal particles are usually used as a substrate, with the help of a surface plasmon generated on a rough metal surface after excitation, a Raman signal of an adsorbent molecule is enhanced, and an issue of low sensitivity of a conventional Raman spectrometer is solved. A Raman signal of a molecule located in a metal particle nanogap (called “a hotspot”) can be further enhanced due to a plasmon coupling effect. The present disclosure provides a new testing thought for the gene damage effector molecule based on an SERS probe testing technology of ligand capture recognition and a Raman molecule label (a molecule with a large Raman scattering cross section, serving as a reporting molecule after being adsorbed to a metal surface).

Compared with an existing method, the present disclosure has the following beneficial effects during impurity genotoxicity assessment:

The method for assessing the impurity genotoxicity through the cell-based sensor based on surface-enhanced Raman scattering established in the present disclosure has good testing universality, high reliability, small impurity consumption and other advantages, which is conducive to pushing assessment on genotoxic impurities during a pharmaceutical research and development. Specifically, the human liver cell has a large quantity of metabolic enzyme systems, can simulate the vivo environment to the greatest degree, avoids some GTI omissions needing metabolic activation, and reduces the impurity consumption; the common effector molecule can respond to various types of gene damages at the same time and effectively improves the genotoxicity assessment speed; and the hotspot is established in situ on a cell by using local overexpression of the effector molecule after the gene damage, so as to implement SERS sensitivity testing, pre-establishing an SERS substrate of a complicated enhancement mechanism in vitro is avoided, and process complexity is reduced.

The organism gene damage and repair usually occur within a finite time, and in-situ real-time testing provides the instant response information after the living cell gene damage, omits the complicated post-processing process and can effectively reduce the false negative rate or the false positive rate.

### BRIEF DESCRIPTION OF THE DRAWINGS

FIG. 1 is a SERS probe preparation line.

FIG. 2 is a spectrum diagram of ultraviolet absorbance of GNP, GPM and Anti γH2AX@GPMT in a SERS probe preparation process.

FIG. 3 is a Zeta potential of GNP, GPM and Anti γH2AX@GPMT in an SERS probe preparation process.

FIG. 4 is a spectrum diagram of Raman scattering of 4-MBN, GNP and Anti γH2AX@GPMT in a SERS probe preparation process.

FIG. 5 is a transmission electron microscope scanning diagram of Anti γH2AX@GPMT of a SERS probe.

FIG. 6 is a concentration-Raman signal response curve of Anti γH2AX@GPMT.

FIG. 7, A is a Raman signal response curve of adding γH2AX of different concentrations into 0.0125 nM Anti γH2AX@GPMT and performing incubation for 30 min; and B is a fitting standard curve after a quotient logarithm of a test group signal and a control group signal in A.

FIG. 8 is a calibrated Raman signal of adding different impurities of 100 ng/mL into 0.0125 nM Anti γH2AX@GPMT and performing incubation for 30 min.

FIG. 9 is a cell viability of incubating an L02 cell for 24 h in GNP and Anti γH2AX@GPMT.

FIG. 10 is a γH2AX immunofluorescence diagram of incubating an L02 cell for 24 h in GNP and Anti γH2AX@GPMT.

FIG. 11 is a cellular uptake dark field imaging diagram of incubating an L02 cell for 4 h in 0.05 nM Anti γH2AX@GPMT.

FIG. 12 is a Raman signal mapping imaging diagram of incubating a cell-based sensor for 24 h in different impurities.

FIG. 13 is a Raman reporting molecule structural formula of 4-MBN, 4-MBA and 4-MPBA.

FIG. 14 is a cellular uptake dark field imaging diagram of incubating an L02 cell for 4 h by a 0.05 nM NLS modified SERS probe.

FIG. 15 is a cellular uptake dark field imaging diagram of incubating HepG2 and Hepa1-6 cells for 4 h in 0.05 nM Anti γH2AX@GPMT.

### DETAILED DESCRIPTION

The technical solutions of the present disclosure will be described clearly and completely below with reference to embodiments. The described embodiments are merely some but not all of embodiments of the present disclosure. All other embodiments obtained by a person of ordinary skill in the art based on the embodiments of the present disclosure without making creative efforts fall within the protection scope of the present disclosure.

### Embodiment 1

### 1. Instrument and Reagent

### 1.1 Instrument

A confocal inverted microscope fluorescent bright and dark field imaging Raman spectrometer (Beijing Zolix, equipped with an Olympus® LUCPLFLN40X type long-focus achromatism objective lens, a Lumenera® INFINITY 3-1 type camera, a 3-scale optical grating, a 3-channel laser light source, a fluorescent light source, a filter and the like, capable of performing bright field and dark field imaging and Raman spectrum testing and performing mapping imaging), a constant temperature incubator, a microplate reader, an ultraviolet spectrophotometer, a transmission electron microscope, an ultrapure water system and the like.

### Reagent

Chlorauric acid tetrahydrate (HAuCl₄·4H₂O), trisodium citrate, SH-PEG-NH₂ (MW2000), 4-mercaptobenzonitrile (4-MBN), glutaraldehyde (25%), PBS, bovine serum albumin (BSA), TAT, an Anti-γH2AX (phospho S139) antibody, methyl methanesulfonate (MMS), cis-platinum (cis-Pt), 5-fluorouracil (5-Fu), and N-nitrosodiethylamine (NDEA).

### Establishing of a Probe

### 2.1 Preparation of a SERS Probe

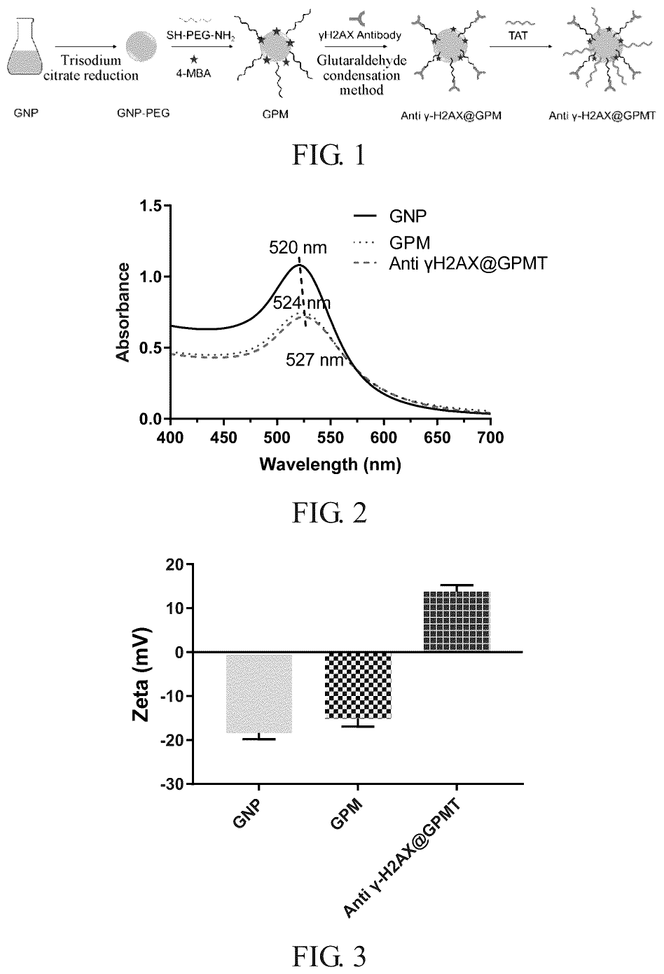

See FIG. 1 for a preparation line of the SERS probe 1.26 g of HAuCl₄·4H₂O is weighed, and dissolved in 100 mL of ultrapure water to prepare a 1% (1 g/100 ml) HAuCl₄ mother solution. 99 mL of ultrapure water is weighed, 1 mL of 1% HAuCl₄ mother solution is added dropwise, 100 mL of 0.01% HAuCl₄ aqueous solution is prepared, stirring and heating are performed to boiling, 4 mL of 1% (1 g/100 mL) trisodium citrate aqueous solution is quickly added, boiling continues for 10 min, cooling is performed to reach a room temperature, and a gold nanoparticle (GNP) solution with a particle diameter being about 16 nm and a concentration being about 5 nM is obtained.

20 mg of SH-PEG-NH₂ is weighed, dissolved in 10 mL of ultrapure water and added into the above gold nanoparticle solution, and stirring is performed for 6 h.

6.75 mg of reporting molecule 4-MBN is weighed, dissolved in 5 mL of ethanol and slowly dropwise added into the above gold nanoparticle solution, stirring is performed for 6 h. then 6000 rpm centrifugation is performed for 10 min to remove a supernatant, a concentrated gold nanoparticle is diluted with ultrapure water to 10 mL, ultrasonic dispersion is performed, and a PEG and 4-MBN modified gold nanoparticle (GPM) is obtained.

0.2 mL of 5% (5 g/100 mL) glutaraldehyde is added into the above GPM, stirring is performed for 2 h at 37° C., 6000 rpm centrifugation is performed for 10 min to remove a supernatant, and ultrasonic dispersion is performed with ultrapure water; and 2 μL of γH2AX antibody is dissolved in 5 ml of aqueous solution, and slowly added into the above glutaraldehyde GPM, incubation is performed for 2 h at 37° C., 6000 rpm centrifugation is performed for 10 min to remove a supernatant, and ultrasonic dispersion is performed with ultrapure water.

0.78 mg of TAT peptide (a molecular weight is 1560) is dissolved in 5 mL of aqueous solution and slowly added into the above solution, stirring is performed for 2 h, 6000 rpm centrifugation is performed for 10 min to remove a supernatant, PBS with 1% BSA (1 g/100 mL) is used for redissolving, ultrasonic dispersion is performed, and the SERS probe Anti γH2AX@GPMT is obtained.

### Representation of the Probe

Preparation and modification processes of the probe are represented by means of an ultraviolet spectrophotometer, a Raman spectrometer and the like, including GNP, GPM and Anti γH2AX@GPMT, and a form, a particle diameter and a potential of Anti γH2AX@GPMT are assessed through a particle size analyzer and a transmission electron microscope.

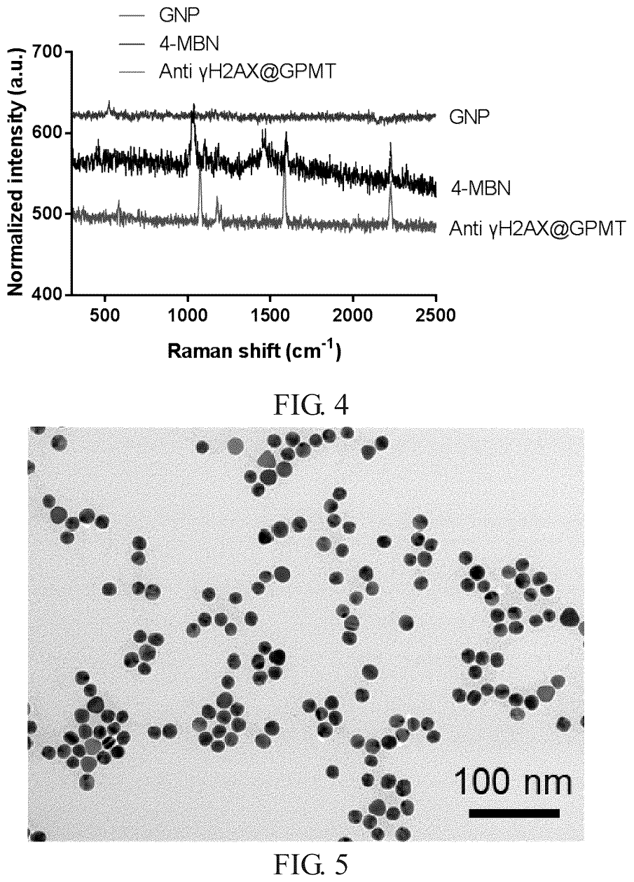

A result shows that an ultraviolet result displays that (FIG. 2) with continuous red shift of a gold nanoparticle SPR peak, it suggests more and more modification groups on the gold nanoparticle, and indirectly proves occurrence of a modification process. A Zeta potential result displays that (FIG. 3) potentials of sodium citrate protected GNP and 4-MBN and PEG modified GPM are all negative values, and after being modified with the TAT peptide, the potentials are reversed and show positive electricity, which is conducive to cell penetrating and nucleus targeting. After testing through the Raman spectrometer (FIG. 4), there is no clear Raman signal in blank GNP, a Raman molecule 4-MBN shows a typical signal peak (1073, 1582 and 2230 cm⁻¹) in a free state, but a response value is low, and a stronger Raman signal is shown in the SERS probe, which proves that an enhancement effect exists on a metal surface. A final SERS probe AntiγH2AX@GPMT shows a stably dispersed spherical nanoparticle under the transmission electron microscope, and a particle diameter is about 15 nm (FIG. 5).

SERS probe aqueous solutions of different concentrations are absorbed with a quartz capillary tube, and the Raman signal is measured under the Raman spectrometer (638 nm laser irradiation, power 29 mW, and Raman shift 2230 cm⁻¹), and a linearity range of testing is determined. Similarly, Raman signals of a free aqueous solution and a SERS probe aqueous solution with the same Raman molecule concentration are measured, and an enhancement factor is calculated according to a formula EF=(Iₛ*N_{f})/(I_{f}*Nₛ), where I represent an intensity of a calibrated Raman signal (a measured value−a background value), N represents the number of molecules, s represents the SERS probe, and f represents a free molecule. A first enhancement effect of the probe is explored. The SERS probe solution and effector molecules (γH2AX) of different concentrations or other intracellular protein molecules (H2AX, GSH, H₂O₂, Arg and the like) are incubated for 30 min, a Raman signal intensity change curve and transmission electron microscope scanning are measured, and a second enhancement effect of inducing the probe by the effector molecules and establishing a hotspot is explored.

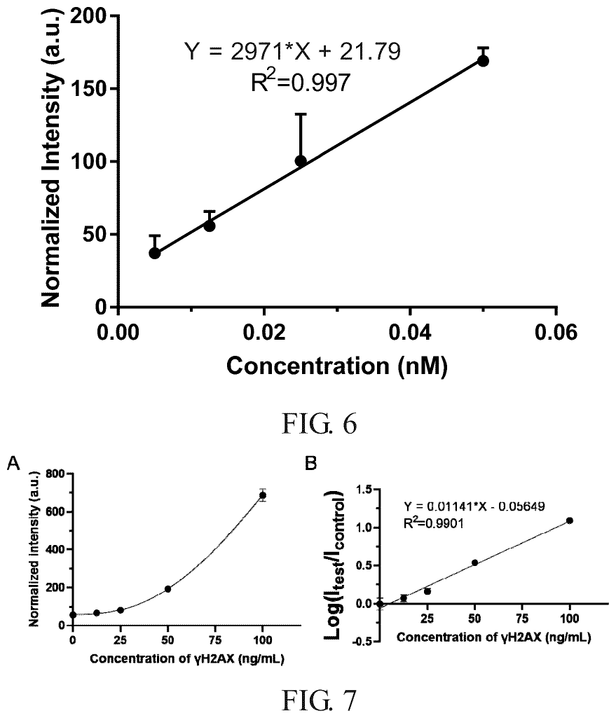

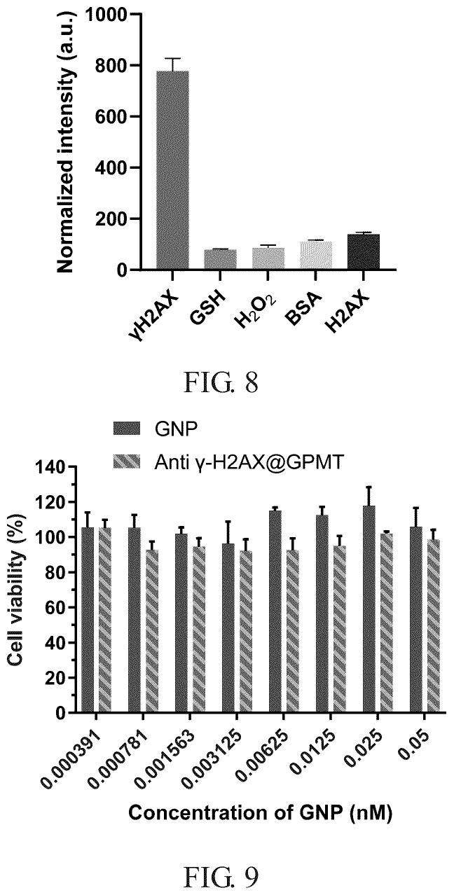

A result shows that within a cell drug administration concentration range, an SERS probe signal-concentration curve has a good linear relation (FIG. 6), and the enhancement factor of the SERS probe is calculated as 1.37×10⁴ according to a calculating formula. Further, a 0.0125 nM SERS probe is taken and incubated with γH2AX peptite of different concentrations in vitro for 30 min respectively, a Raman signal intensity is measured, a result shows that (A in FIG. 7) with increase of a protein concentration, SERS probes are aggregated, with increase of an aggregation degree, the Raman signal is in exponential increase, it suggests that a hotspot may be formed so as to further enhance the signal, after incubation in 100 ng/ml γH2AX, the enhancement factor of the Raman signal may reach 2.1×10⁵, a detection limit is expected to decrease remarkably, and a detection sensitivity is improved. On the basis of A in FIG. 7, through logarithmic fitting of quotients of signals in the test group and the control group, a standard curve with a good linearity is obtained (see B in FIG. 7), which is used for calculating a fold of induction of the effector molecules and assessing genotoxicity. Further, the 0.0125 nM SERS probe is taken and incubated with 100 ng/mL of GSH, H₂O₂, BSA and H2AX in vitro for 30 min respectively, the Raman signal is measured, a result suggests that (FIG. 8) merely γH2AX can cause remarkable enhancement of the SERS probe signal, and it shows that the probe has a detection specificity.

### Establishing of the Cell-Based Sensor

### 3.1 Preparation of the Cell-Based Sensor

A human liver cell line L02 is cultured in DMEM containing 10% fetal calf serum, and put in a 37° C. incubator containing 5% CO₂, and a sterile operation is performed in an entire process. When the cells grow to a logarithmic phase, L02 is inoculated into a 24-well plate containing a cell slide according to a density of 5000 cells/well, after being attached to a wall, a culture medium is replaced by a SERS probe culture medium with a concentration being 0.05 nM, and incubation is performed for 4 h.

### Assessment of the Cell-Based Sensor

Cell activity: whether GNP and Anti γH2AX@GPMT have toxicity to the L02 cells is explored through an in-vitro cytotoxicity test and a DNA damage test. The L02 cells in the logarithmic phase are collected, and a cell suspension of an appropriate concentration is prepared with the DMEM culture medium containing 10% fetal calf serum.
(1) MTT test: 100 μL is taken and inoculated into a 96-well culture plate, and culturing is performed for 12 h so as to make the cells be attached to a wall. Setting the test group: a culture medium of GNP or Anti γH2AX@GPMT of different concentrations is added into each well. Setting the control group separately: the cells and a blank culture medium are added, and a blank group: no cell is added and merely a culture medium is added. Incubation is performed in 5% CO₂ at 37° C. for 24 h, the 96-well plate is taken out, 10 μL of the MTT solution (5 mg/ml) is added into each well, incubation away from light is continued for 4 h at 37° C. in a cell incubator containing 5% CO₂, an absorbance of each well is measured under 492 nm by using a microplate reader, and a cell viability is calculated according to the following formula:

$$cell viability ( \% ) = \frac{{A}_{S} - {A}_{b}}{{A}_{c} - {A}_{b}} \times 100 \%$$

where, Aₛ=absorbance of the test group, A_{b}=absorbance of the blank group, and A_{c}=absorbance of the control group.
(2) γH2AX immunofluorescence assay: 1 mL is taken and inoculated into a 6-well culture plate, and culturing is performed for 12 h to make the cells be attached to a wall Setting the test group: the culture medium of GNP or Anti γH2AX@GPMT of different concentrations is added into each well; setting the control group separately: the cells and the blank culture medium are added; and culturing is performed for 24 h in 5% CO₂ at 37° C. The plate is taken out; the cells are fixed for 15 min with 4% (4 g/100 mL) paraformaldehyde; room temperature permeabilization is performed for 20 min with 0.5% Triton X-100 (0.5 ml/100 ml PBS); 2% (2 g/100 ml) BSA is added dropwise, and room temperature blocking is performed for 30 min; a sufficient quantity of primary antibody Anti-γ H2AX antibody diluted with a blocking buffer according to a ratio of 1:200 is added dropwise into each well; incubation is performed at 4° C. overnight; a fluorescent secondary antibody Goat Anti-Rabbit IgG H&L (Alexa Fluor® 488) diluted according to a ratio of 1:1000 is added dropwise, and room temperature incubation is performed for 1 h; and DAPI is added dropwise, incubation away from light is performed for 5 min, and nucleus staining is performed on a specimen. Immersion cleaning is performed with PBS or PBST when each step is completed, and then a collected image is observed under a fluorescence microscope.

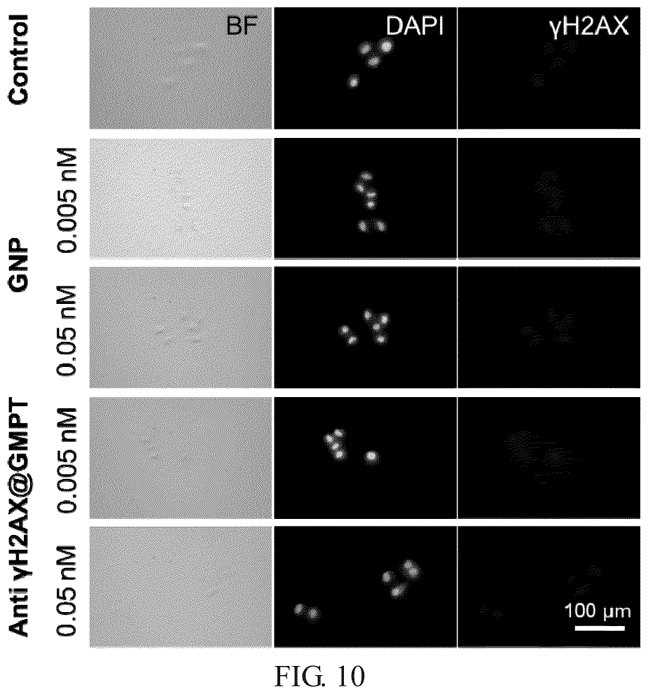

An MTT result shows that (FIG. 9) the GNP and the Anti γH2AX@GPMT have small toxicity to the L02 cells, each cell viability exceeds 90%, and there is no concentration-dependency, which suggests no existence of clear cytotoxicity. A γH2AX immunofluorescence assay result shows (FIG. 10) that γH2AX is negative when the probe is in 0.05 nM cell drug administration concentration, which suggests that the GNP and the Anti γH2AX@GPMT also have no clear toxicity on a DNA damage level.

Cellular uptake: a cell-based sensor culture medium after incubation is absorbed, cleaning is performed with PBS, and a cellular uptake condition of the SERS probe is observed under a dark field microscope according to a gold nanoparticle dark field scattering imaging function.

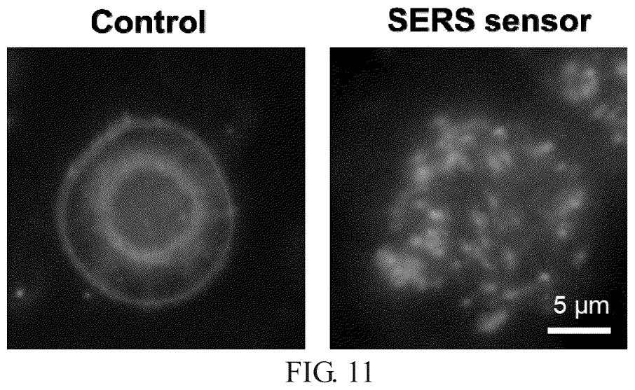

A result shows (FIG. 11) that compared with a blank cell, an appropriate quantity of SERS probes is distributed in the cell-based sensor, which is conducive to assessment on impurity genotoxicity in a next step.

### Assessment on the Impurity Genotoxicity

The cell-based sensor culture medium is replaced by a culture medium containing impurities of different concentrations, continuous incubation is performed for 24 h, then the slide is taken out and put in a confocal inverted microscope bright and dark field imaging Raman spectrometer to be tested, the Raman signals of the test group and the control group are compared, and the impurity genotoxicity is assessed.

Methyl methanesulfonate (MMS), a salicylic acid (SA), 5-fluorouracil (5-Fu) and N-nitrosodiethylamine (NDEA) are selected respectively as genotoxic impurities of different structure types to be tested. A drug administration concentration is set as a concentration under a proximate cytotoxicity action, namely, MMS (50 μg/ml), SA (50 μg/ml), S-Fu (0.1 μg/ml) and NDEA (500 μg/ml). During Raman mapping imaging, a center point of a cell nucleus is used as a circle center, 2 μm is a step length, a 20×20 μm square is scanned (2230 cm⁻¹ is used as a characteristic signal peak), and when signal intensity is calculated, an average value is obtained with a lattice signal covering a cell nucleus area. This signal is transformed into an effector molecule concentration through a standard curve, a concentration ratio of the effector molecules of the test group and the control group is calculated, namely, the fold of induction (FI), when FI is greater than 1.5, it is judged as a genotoxic impurity of this type, and when it is smaller than or equal to 1.5, it is judged as not the genotoxic impurity of this type.

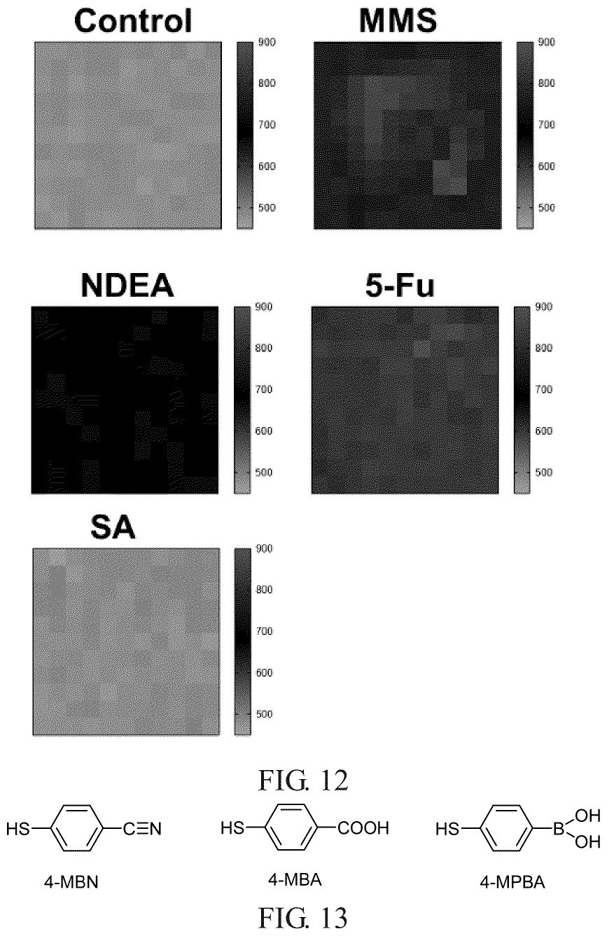

A result shows (FIG. 12 & Table 1) that it is known that the genotoxic impurities are tested through the sensor, and each FI value is greater than 1.5, which suggests that genotoxicity really exists; and it is known that each FI value of a non-genotoxic impurity is smaller than 1.5, which suggests that this method can effectively assess the impurity genotoxicity.

TABLE 1
FI value data of each impurity tested through a cell-based sensor

| | Control | MMS | NDEA | 5-Fu | SA |
|---|---|---|---|---|---|
| FI | 1.00 | 4.81 | 3.59 | 5.19 | 1.02 |

### Embodiment 2

### 1. Instrument and Reagent

### 1.1 Instrument

A confocal inverted microscope fluorescent bright and dark field imaging Raman spectrometer (Beijing Zolix, equipped with an Olympus® LUCPLFLN40X type long-focus achromatism objective lens, a Lumenera® INFINITY 3-1 type camera, a 3-scale optical grating, a 3-channel laser light source, a fluorescent light source, a filter and the like, capable of performing bright field and dark field imaging and Raman spectrum testing and performing mapping imaging), a constant temperature incubator, an ultrapure water system and the like.

### Reagent

Chlorauric acid tetrahydrate (HAuCl₄·4H₂O), trisodium citrate, SH-PEG-NH₂ (MW2000), 4-mercaptobenzonitrile (4-MBN), 4-mercaptobenzoic acid (4-MBA), 4-mercaptophenylboronic acid (4-MBN), a glutaraldehyde (25%), PBS, bovine serum albumin (BSA), TAT, NLS, and an Anti-γH2AX (phospho S139) antibody.

### Selective Optimization of the Raman Molecule

The 4-MBN, the 4-MBA and the 4-MPBA are similar in structure (FIG. 13), use a sulfhydryl group as a group to be connected with the gold nanoparticle, form a Raman scattering cross section through a benzene ring and represent a characteristic Raman signal peak through different substituent groups, so the above three have been widely applied. Therefore, the present disclosure believes that the selected above three types of Raman molecules have proximate signal reporting functions without affecting a physicochemical property of the cell-based sensor, which may be used as the Raman molecules used in the present disclosure.

### Selective Optimization of the Cell-Penetrating Peptide

Under a preparation condition of Embodiment 1, the TAT peptide is replaced by an equal molar weight of NLS peptide for exploring cellular uptake of the probe.

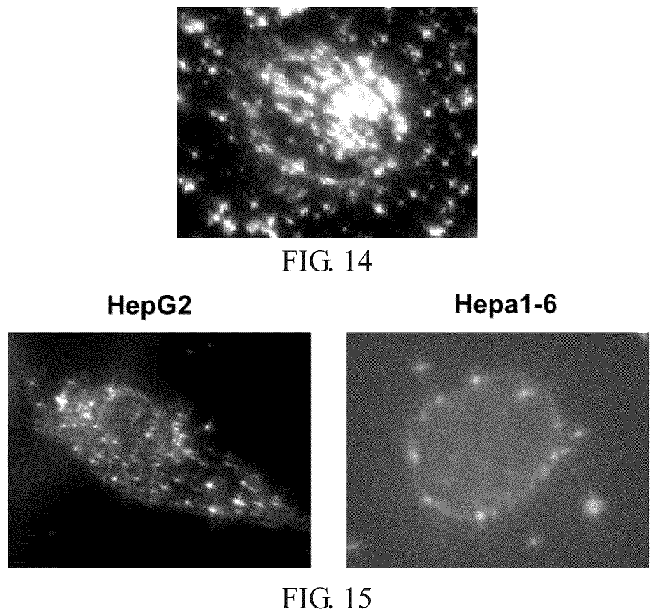

A result shows (FIG. 14) that within 4 h of cellular uptake, the NLS modified SERS probes are massively aggregated in a cell nucleus, which causes a large background interference to subsequent probe aggregation triggered by genotoxic impurity induced γH2AX in the present disclosure. This may be because a nucleus targeting capability of the NLS is stronger, its modification ratio and a cellular uptake concentration and time need to be further optimized, so as to meet a testing demand, and improve a signal contrast.

### Selective Optimization of a Cell-Based Sensor Carrier

Under a preparation condition of the cell-based sensor of Embodiment 1, the L02 cells are replaced by HepG2 and Hepa1-6 cells for exploring cellular uptake of the probe.

A result shows (FIG. 15) that within 4 h of cellular uptake, the quantity of probes entering the nucleus in HepG2 and Hepa1-6 cellular uptake is small, which may be due to high metabolic activity and strong efflux transport capacity of tumor cells, leading to fewer probes contained in the cell-based sensor, sensitive genotoxicity testing is difficult to implement, and an incubation concentration and time of the probe need to be further optimized, so as to meet a testing demand and improve a signal sensitivity.

1. A cell-based sensor based on surface-enhanced Raman scattering, wherein the cell-based sensor is established by guiding a surface-enhanced Raman scattering probe into a human liver cell line;
the surface-enhanced Raman scattering probe is prepared by using a gold nanoparticle as a testing substrate, a gene damage effector molecule antibody as a recognition unit, a Raman molecule as a reporting unit, SH-PEG-NH₂ as a stable chain and a cell-penetrating peptide as an auxiliary penetration unit;
the gene damage effector molecule antibody is a γH2AX antibody;
the Raman molecule includes at least one of 4-cyanothiophenol, 4-mercaptobenzoic acid or 4-mercaptophenylboronic acid;
the human liver cell line is at least one of a human liver cell L02, a human liver cancer cell HepG2 or a human liver cancer cell Hepa1-6; and
the cell-penetrating peptide includes at least one of TAT or NLS.

2. The cell-based sensor according to claim 1, wherein the surface-enhanced Raman scattering probe is prepared by the following steps:
step (1): preparing a gold nanoparticle solution through a trisodium citrate reduction method;
step (2): modifying the gold nanoparticle by SH-PEG-NH₂, specifically, SH-PEG-NH₂ is added into the gold nanoparticle solution prepared in step (1) for a stirring reaction to obtain an SH-PEG-NH₂ modified gold nanoparticle solution;
step (3): modifying the gold nanoparticle by the Raman molecule, specifically, a Raman molecule solution is slowly added into the gold nanoparticle solution prepared in step (2) for a stirring reaction, then centrifugation is performed to remove a supernatant, and ultrapure water is added for uniform dispersion to obtain an SH-PEG-NH₂ and Raman molecule modified gold nanoparticle solution;
step (4): modifying the gold nanoparticle by the gene damage effector molecule antibody, specifically, a 5% glutaraldehyde solution is added into the gold nanoparticle solution prepared in step (3) for a stirring reaction, then centrifugation is performed to remove a supernatant, ultrapure water is added for uniform dispersion to obtain a glutaraldehyde gold nanoparticle solution, then a gene damage effector molecule antibody aqueous solution is added for incubation, then centrifugation is performed to remove a supernatant, and ultrapure water is added for uniform dispersion to obtain a gene damage effector molecule antibody modified gold nanoparticle solution; and
step (5): modifying the gold nanoparticle by the cell-penetrating peptide, specifically, the cell-penetrating peptide is added into the gold nanoparticle solution prepared in step (4) for a stirring reaction, then centrifugation is performed to remove a supernatant, PBS containing 1% BSA is used for redissolving, and uniform dispersion is performed to obtain a surface-enhanced Raman scattering probe.

3. The cell-based sensor according to claim 1, wherein a particle diameter of the gold nanoparticle is 10 to 50 nm.

4. The cell-based sensor according to claim 2, wherein a process of preparing the gold nanoparticle solution by using the trisodium citrate reduction method in step (1) is:
a 0.01% HAuCl₄ aqueous solution is heated to boiling, a 1% trisodium citrate aqueous solution is quickly added, and
boiling is performed for 7 to 10 min, wherein a volume ratio of the 0.01% HAuCl₄ aqueous solution to the 1% trisodium citrate aqueous solution is 20:1 to 100:1.

5. The cell-based sensor according to claim 2, wherein
a molecular weight of the SH-PEG-NH₂ in step (2) is 2000 to 5000; a molar ratio of the gold nanoparticle to the SH-PEG-NH₂ is 1:1×10³ to 1:2×10⁶;
a molar ratio of the gold nanoparticle to the Raman molecule in step (3) is 1:1×10³ to 1:1×10⁶;
a molar ratio of the gold nanoparticle to the glutaraldehyde in step (4) is 1:1×10³ to 1:2×10⁶; a charge ratio of the gold nanoparticle to the gene damage effector molecule antibody is 5 pmol:2 μL to 5 nmol:2 μL; and
a molar ratio of the gold nanoparticle to the cell-penetrating peptide in step (5) is 1:1×10² to 1×1:10⁵.

6. The cell-based sensor according to claim 2, wherein time of each stirring reaction in step (2), step (3) and step (5) is 5 to 10 hours independently; and time of the stirring reaction in step (4) is 1 to 3 hours, and an incubation condition is incubation for 1 to 3 hours at 25° C. to 38° C.

7. A method comprising assessing genotoxic impurity levels with the cell-based sensor of claim 1.

8. A genotoxic impurity assessment method based on surface-enhanced Raman scattering, wherein the method uses a gold nanoparticle as a testing substrate, a gene damage effector molecule antibody as a recognition unit, a Raman molecule as a reporting unit, SH-PEG-NH₂ as a stable chain and a cell-penetrating peptide as an auxiliary penetration unit to prepare a surface-enhanced Raman scattering probe; the surface-enhanced Raman scattering probe is guided into a human liver cell line to establish a cell-based sensor; the cell-based sensor is exposed to drug impurities of different DNA damage mechanisms, a Raman signal is detected, and a genotoxicity level of the drug impurities is assessed; and the cell-based sensor is the cell-based sensor according to claim 1.

9. The method according to claim 8, wherein when a gene damage occurs, an effector molecule is overexpressed at the damage, surface-enhanced Raman scattering probes are induced to aggregate to form a hotspot, a surface-enhanced Raman scattering enhanced signal is generated, in-situ real-time monitoring is performed under a Raman microscope, and a genotoxicity level of drug impurities is assessed through intensity change of a Raman signal in a gene damage process.

10. The method according to claim 8, wherein a concentration of the drug impurities is supposed to ensure that a cell viability of the cell-based sensor is 75% or above; when the Raman signal is detected, an excitation wavelength of a light source of the Raman spectrometer is 638 nm, and the detected signal uses a peak value of a characteristic Raman peak after 1800 cm⁻¹ of a Raman shift; and the detected signal is transformed to an effector molecule concentration through a standard curve, a concentration ratio of effector molecules of a test group and a control group is calculated, namely, a fold of induction (FI), when FI is greater than 1.5, it is judged as a DNA-damage type genotoxic impurity, and when FI is smaller than or equal to 1.5, it is judged as a non-DNA-damage type genotoxic impurity.

11. The cell-based sensor according to claim 2, wherein the particle diameter of the gold nanoparticle is 10 to 50 nm.

12. A method comprising assessing genotoxic impurity levels with the cell-based sensor of claim 2.

13. A method comprising assessing genotoxic impurity levels with the cell-based sensor of claim 4.

14. A method comprising assessing genotoxic impurity levels with the cell-based sensor of claim 5.

15. A method comprising assessing genotoxic impurity levels with the cell-based sensor of claim 6.

16. A genotoxic impurity assessment method based on surface-enhanced Raman scattering, wherein the method uses the gold nanoparticle as the testing substrate, the gene damage effector molecule antibody as the recognition unit, the Raman molecule as the reporting unit, SH-PEG-NH₂ as the stable chain and the cell-penetrating peptide as the auxiliary penetration unit to prepare the surface-enhanced Raman scattering probe; the surface-enhanced Raman scattering probe is guided into the human liver cell line to establish the cell-based sensor; the cell-based sensor is exposed to drug impurities of different DNA damage mechanisms, the Raman signal is detected, and the genotoxicity level of the drug impurities is assessed; and the cell-based sensor is the cell-based sensor according to claim 2.

17. A genotoxic impurity assessment method based on surface-enhanced Raman scattering, wherein the method uses the gold nanoparticle as the testing substrate, the gene damage effector molecule antibody as the recognition unit, the Raman molecule as the reporting unit, SH-PEG-NH₂ as the stable chain and the cell-penetrating peptide as the auxiliary penetration unit to prepare the surface-enhanced Raman scattering probe; the surface-enhanced Raman scattering probe is guided into the human liver cell line to establish the cell-based sensor; the cell-based sensor is exposed to drug impurities of different DNA damage mechanisms, the Raman signal is detected, and the genotoxicity level of the drug impurities is assessed; and the cell-based sensor is the cell-based sensor according to claim 4.

18. A genotoxic impurity assessment method based on surface-enhanced Raman scattering, wherein the method uses the gold nanoparticle as the testing substrate, the gene damage effector molecule antibody as the recognition unit, the Raman molecule as the reporting unit, SH-PEG-NH₂ as the stable chain and the cell-penetrating peptide as the auxiliary penetration unit to prepare the surface-enhanced Raman scattering probe; the surface-enhanced Raman scattering probe is guided into the human liver cell line to establish the cell-based sensor; the cell-based sensor is exposed to drug impurities of different DNA damage mechanisms, the Raman signal is detected, and the genotoxicity level of the drug impurities is assessed; and the cell-based sensor is the cell-based sensor according to claim 5.

19. A genotoxic impurity assessment method based on surface-enhanced Raman scattering, wherein the method uses the gold nanoparticle as the testing substrate, the gene damage effector molecule antibody as the recognition unit, the Raman molecule as the reporting unit, SH-PEG-NH₂ as the stable chain and the cell-penetrating peptide as the auxiliary penetration unit to prepare the surface-enhanced Raman scattering probe; the surface-enhanced Raman scattering probe is guided into the human liver cell line to establish the cell-based sensor; the cell-based sensor is exposed to drug impurities of different DNA damage mechanisms, the Raman signal is detected, and the genotoxicity level of the drug impurities is assessed; and the cell-based sensor is the cell-based sensor according to claim 6.
